(19) United States
(12) Patent Application Publication
(10) Pub. No.: US 2007/0276207 A1
(43) Pub. Date: Nov. 29, 2007

(54) Polymeric Materials Incorporating a Ph Indicator Dye

(76) Inventors: Donald Eagland, West Yorkshire (GB); Nicholas John Crowther, West Yorkshire (GB)

Correspondence Address: FAY SHARPE LLP, 1100 SUPERIOR AVENUE, SEVENTH FLOOR, CLEVELAND, OH, 44114, US

(21) Appl. No.: 10/580,383

(22) PCT Filed: Nov. 19, 2004
(86) PCT No.: PCT/GB04/04869

§ 371 Date: Mar. 20, 2007

(30) Foreign Application Priority Data
Nov. 22, 2003 (GB) 0327226.7

(51) Int. Cl. A61B 5/145

(52) U.S. Cl. 600/309

(57) ABSTRACT

The pH of a wound is assessed using a hydrogel film which changes colour in dependence upon pH. Suitably, the hydrogel film incorporates an indicator, across substantially its entire extent so that the film may be used to monitor pH across a wound. Wound pH information may be used to facilitate selection of the appropriate treatment to which the wound should be subjected. Suitably, the hydrogel film is incorporated into a dressing.

This invention relates to polymeric materials and particularly, although not exclusively, relates to materials in the form of hydrogels. Preferred embodiments relate to the use of such materials in assessing the pH of a substrate, for example a body tissue such as a wound to facilitate a medical diagnosis and appropriate treatment of the wound.

The treatment of body tissues, for example wounds to human or animal bodies can be problematic due to difficulties in assessing characteristics of the wound, for example the pH of exudates, to facilitate detailed assessment of a wound, correct diagnosis and hence selection of an appropriate treatment.

It is an object of one embodiment of the present invention to address the aforesaid problem.

It is an object of other embodiments to provide polymeric materials and/or methods which may be of utility, for example in medical and other applications.

According to a first aspect of the invention, there is provided a method of assessing the pH of a substrate or environment, the method comprising contacting the substrate with a test material or introducing the test material into an environment, wherein said test material is arranged to change colour according to pH.

Said test material preferably comprises a polymeric material. Such a polymeric material may be naturally-occurring or synthetic. More preferably, it comprise a hydrogel. A said hydrogel may be defined as a cross-linked, water insoluble, water containing material.

Said hydrogel suitably contains at least 50 wt %, preferably at least 60 wt %, more preferably at least 70 wt %, especially at least 80 wt % water. The amount of water may be 95 wt % or less. In a preferred embodiment, the amount of water is in the range 90 to 95 wt %. The level of water may be determined by any suitable means, for example by thermogravimetric analysis.

A said hydrogel may comprise a natural or synthetic polysaccharide, polyacrylate, polyacrylamide, or cross-linked polyvinylalcohol, polyvinylacetate, polyalkylene glycols, for example propylene glycols (and copolymers of the aforementioned) and collagen (and any component thereof).

Said test material preferably comprises a carrier means and an indicator means arranged to change colour according to pH. Said carrier means and said indicator means may be covalently bonded to one another or said carrier means and indicator means may be associated with one another in another way. For example, said indicator means may be impregnated in said carrier means and, suitably, trapped therein in a matrix defined by said carrier means. Said indicator means is preferably substantially uniformly dispersed throughout the carrier means. Preferably, said test material is such that said indicator means does not leach therefrom to any significant degree, in use.

Preferably, the ratio of the concentration (in moles) of indicator means in said test material at least 1 minute, preferably at least 5 minutes, especially at least 1 hour after initial contact with said substrate compared to the s concentration (in moles) at the time of initial contact with said substrate is at least 0.9, preferably at least 0.95, more preferably at least 0.99, especially about 1.

Said test material suitably includes at least 0.01 wt %, preferably at least 0.05 wt %, more preferably at least 0.08 wt % of said indicator means, wherein the weight of said indicator means is measured on a dry weight basis. Said test material suitably includes less than 3 wt %, preferably less than 1 wt %, more preferably less than 0.5 wt %, especially less than 0.2 wt % of said indicator means when assessed as aforesaid.

Said carrier means preferably makes up at least 90 wt % of said test material when the weight of water in said test material is excluded.

Said carrier means may comprise a natural or synthetic polymer or a residue thereof in the event that said indicator means is covalently bonded to the carrier means. Polysaccharides and collagen (and any component thereof) are examples of suitable natural polymers. Synthetic polymers include optionally cross-linked poly(vinyl alcohol), poly (vinyl acetate), polyalkylene glycols, polyacrylates, polyacrylamides and copolymers of the aforesaid, for example poly(vinyalcohol) copolymers.

Said indicator means may comprise a natural or synthetic material or a residue thereof in the event said indicator means is covalently bonded to said carrier means. Said indicator means may be any pH sensitive indicator which is compatible with the carrier means such that it may be associated therewith, either by being covalently bonded thereto or impregnated therein. Said indicator means is suitably sensitive at least within the range pH 4-8, preferably at least within the range 2 to 10, more preferably at least within the range 1 to 14. Suitably said indicator means has an accuracy of at least 1 pH unit, preferably at least 0.75 pH unit, especially at least 0.5 pH unit.

A said indicator means may be covalently bonded to a said carrier means in a condensation reaction, for example an aldol condensation or an acetylation reaction. Other reactions may be used in dependence upon the functional groups available.

Conventional indicators may be covalently bonded to the carrier means in some situations.

Advantageously, indicator means of the type described, for example Universal indicator, can be associated with said carrier means for use in the method, without being covalently bonded to the carrier means.

A polymeric material which may itself act as an indicator means and thereby be arranged to change colour according to pH may comprise:

(a) a first polymeric material having a repeat unit of formula 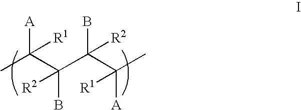
wherein A and B are the same or different, are selected from optionally-substituted aromatic and heteroaromatic groups and at least one comprises a relatively polar atom or group and R¹ and R² independently comprise relatively non-polar atoms or groups; or

(b) a first polymeric material prepared or preparable by providing a compound of general formula

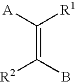

wherein A, B, R¹ and R² are as described above, in an aqueous solvent and causing the groups C═C in said compound to react with one another to form said first polymeric material.

Preferably, in said first polymeric material, A and B are the same or different, are selected from optionally-substituted aromatic and heteroaromatic groups and at least one comprises a relatively polar atom or group and R¹ and R² independently comprise relatively non-polar atoms or groups.

A and/or B could be multi-cyclic aromatic or heteroaromatic groups. Preferably, A and B are independently selected from optionally-substituted five or more preferably six-membered aromatic and heteroaromatic groups]. Preferred heteroatoms of said heteroaromatic groups include nitrogen, oxygen and sulphur atoms of which oxygen and especially nitrogen, are preferred. Preferred heteroaromatic groups include only one heteroatom. Preferably, a or said heteroatom is positioned furthest away from the position of attachment of the heteroaromatic group to the polymer backbone. For example, where the heteroaromatic group comprises a six-membered ring, the heteroatom is preferably provided at the 4-position relative to the position of the bond of the ring with the polymeric backbone.

Preferably, A and B represent different groups. Preferably, one of A or B represents an optionally-substituted aromatic group and the other one represents an optionally-substituted heteroaromatic group. Preferably A represents an optionally-substituted aromatic group and B represents an optionally-substituted heteroaromatic group especially one including a nitrogen heteroatom such as a pyridinyl group.

Unless otherwise stated, optionally-substituted groups described herein, for example groups A and B, may be substituted by halogen atoms, and optionally substituted alkyl, acyl, acetal, hemiacetal, acetalalkyloxy, hemiacetalalkyloxy, nitro, cyano, alkoxy, hydroxy, amino, alkylamino, sulphinyl, alkylsulphinyl, sulphonyl, alkylsulphonyl, sulphonate, amido, alkylamido, alkylcarbonyl, alkoxycarbonyl, halocarbonyl and haloalkyl groups. Preferably, up to 3, more preferably up to 1 optional substituents may be provided on an optionally substituted group.

Unless otherwise stated, an alkyl group may have up to 10, preferably up to 6, more preferably up to 4 carbon atoms, with methyl and ethyl groups being especially preferred.

Preferably, A and B each represent polar atoms or group that is, there is preferably some charge separation in groups A and B and/or groups A and B do not include carbon and hydrogen atoms only.

Preferably, at least one of A or B includes a functional group which can undergo a condensation reaction, for example on reaction with a said carrier means to define a test material wherein a said carrier means and a said indicator means are covalently bonded to one another. Preferably, A includes a said functional group which can undergo a condensation reaction.

Preferably, one of groups A and B includes an optional substituent which includes a carbonyl or acetal group with a formyl group being especially preferred. The other one of groups A and B may include an optional substituent which is an alkyl group, with an optionally substituted, preferably unsubstituted, C₁₋₄ alkyl group, for example a methyl group, being especially preferred.

Preferably, A represents a group, for example an aromatic group, especially a phenyl group, substituted (preferably at the 4-position relative to polymeric backbone when A represents an optionally-substituted phenyl group) by a formyl group or a group of general formula 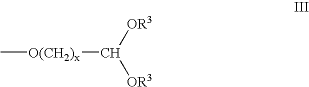
where x is an integer from 1 to 6 and each R³ is independently an alkyl or phenyl group or together form an alkalene group.

Preferably, B represents an optionally-substituted heteroaromatic group, especially a nitrogen-containing heteraromatic group, substituted on the heteroatom with a hydrogen atom or an optionally-substituted alkyl or aralkyl group. More preferably, B represents a group of general formula 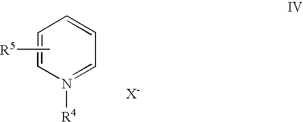
wherein R⁴ represents a hydrogen atom or an optionally-substituted alkyl or aralkyl group, R⁵ represents a hydrogen atom or an alkyl group and X⁻ represents a strongly acidic ion.

Preferably, R¹ and R² are independently selected from a hydrogen atom or an optionally-substituted, preferably unsubstituted, alkyl group. Preferably, R¹ and R² represent the same atom or group. Preferably, R¹ and R² represent a hydrogen atom.

Preferred first polymeric materials may be prepared from any of the following monomers by the method described in WO98/12239 and the content of the aforementioned document is incorporated herein by reference:

α-(p-formylstyryl)-pyridinium, γ-(p-formylstyryl)-pyridinium, α-(m-formylstyryl)-pyridinium, N-methyl-α-(p-formylstyryl)-pyridinium, N-methyl-β-(p-formylstyryl)-pyridinium, N-methyl-α-(m-formylstyryl)-pyridinium, N-methyl-α-(o-formylstyryl) -pyridinium, N-ethyl-α-(p-formylstyryl)-pyridinium, N-(2-hydroxyethyl)-α-(p-formylstyryl)-pyridinium, N-(2-hydroxyethyl)-γ-(p-formylstyryl)-pyridinium, N-allyl-α-(p-formylstyryl)-pyridinium, N-methyl-γ-(p-formylstyryl) -pyridinium, N-methyl-γ-(m-formylstyryl)-pyridinium, N-benzyl-α-(p-formylstyryl)-pyridinium, N-benzyl-γ-(p-formylstyryl)-pyridinium and N-carbamoylmethyl-γ-(p-formylstyryl)-pyridinium. These quaternary salts may be used in the form of hydrochlorides, hydrobromides, hydroiodides, perchlorates, tetrafluoroborates, methosulfates, phosphates, sulfates, methane-sulfonates and p-toluene-sulfonates.

Also, the monomer compounds may be styrylpyridinium salts possessing an acetal group, including the following:

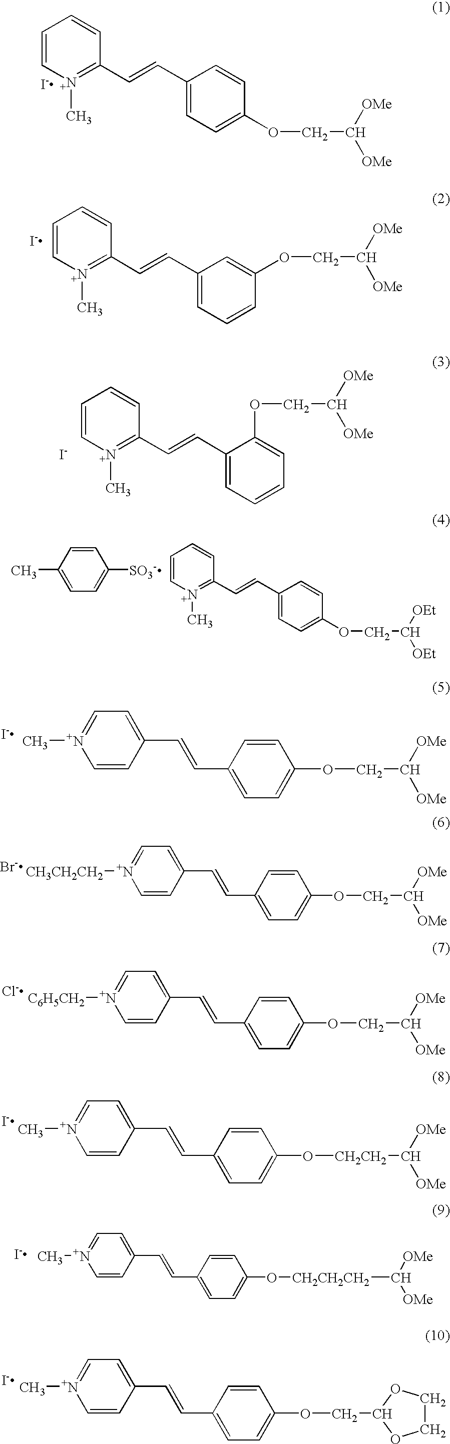

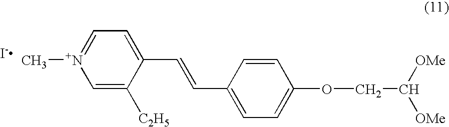

Said first polymeric material may be of formula 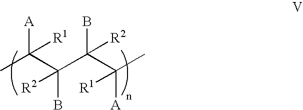
wherein A, B, R¹ and R² are as described above and n is an integer. Integer n is suitably 10 or less, preferably 8 or less, more preferably 6 or less, especially 5 or less. Integer n is suitably at least 1, preferably at least 2, more preferably at least 3.

A preferred test material includes a second polymeric material comprising a third polymeric material which is cross-linked by a cross-linking means. Said second polymeric material may be prepared by selecting a third polymeric material and treating it with a said cross-linking means. Said third polymeric material may include (before being cross-linked as described) functional groups selected from hydroxy, carboxylic acid, carboxylic acid derivatives (e.g. ester) and amine groups. Said third polymeric material preferably includes a backbone comprising, preferably consisting essentially, of carbon atoms. The backbone is preferably saturated. Pendent from the backbone are one or more said functional groups described. Said third polymeric material may have a molecular weight of at least 10,000. Said third polymeric material is preferably a polyvinyl polymer. It may be a copolymer comprising a polyvinyl polymer. Preferred third polymeric materials include optionally substituted, preferably unsubstituted, polyvinylalcohol, polyvinylacetate, polyalkylene glycols, for example polypropylene glycol, and collagen (and any component thereof). Polyvinylalcohol is an especially preferred third polymeric material.

In especially preferred embodiments said second polymeric material includes cross-linked polyvinyl alcohol.

A preferred cross-linking means comprises a chemical cross-linking material. Such a material is preferably a polyfunctional compound having at least two functional groups capable of reacting with functional groups of said third polymeric material. Preferably, said cross-linking means includes one or more of carbonyl, carboxyl, hydroxy, epoxy, halogen or amino functional groups which are capable of reacting with groups present along the polymer backbone or in the polymer structure of the third polymeric material. Preferred cross-linking means include at least two aldehyde groups. Thus, in a preferred embodiment, said second polymeric material includes a material formed by cross-linking a polyvinylalcohol-containing polymer or copolymer using a material having at least two aldehyde groups. Thus, said second polymeric material preferably includes a moiety of formula I. 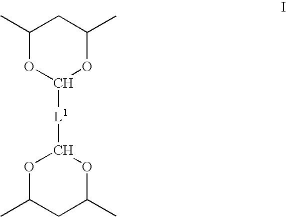
wherein L¹ is a residue of said cross-linking means.

Said cross-linking means preferably comprises said first polymeric material as described above.

Preferably, formation of said second polymeric material from said third polymeric material and said cross-linking means (especially when said cross-linking means comprises said first polymeric material) involves a condensation reaction. Preferably, formation of said second polymeric material involves an acid catalysed reaction. Preferably, said third polymeric material and said cross-linking means include functional groups which are arranged to react, for example to undergo a condensation reaction, thereby to form said second polymeric material.

Said second polymeric material may be prepared by providing a mixture of said third polymeric material and said cross-linking means, especially said first polymeric material described, and causing the two materials to react. Preferably, said mixture includes at least 2 wt %, more preferably at least 3 wt % of said third polymeric material. When the molecular weight of the third polymeric material is relatively low (e.g. 50,000) the maximum amount of said third polymeric material in the mixture may be up to 40 wt %. When the molecular weight of the third polymeric material is higher then the maximum amount may be less, for example up to 30 wt %, or up to 20 wt %. Said mixture may include at least 0.05 wt %, preferably at least 0.1 wt % of said cross-linking means, especially said first polymeric material. The amount of said cross-linking means may be up to 3 wt %.

In the preparation of said second polymeric material, said third polymeric material and said cross-linking means are preferably provided in water. Said mixture may include at least 80 wt %, suitably includes at least 85 wt %, preferably includes at least 90 wt %, water. Said mixture may include other minor components, for example a catalyst, especially an acid, for catalysing the formation of said second polymeric material from said third polymeric material and said cross-linking means.

The ratio of the wt % of said third polymeric material to said cross-linking means used to prepare said second polymeric material is suitably at least 10, preferably at least 15, more preferably at least 19. The ratio may be less than 50, preferably less than 40, especially less than 30.

Said second polymeric material suitably includes a moiety of formula 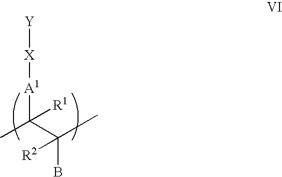
wherein R¹, R² and B are as described above, A¹ represents a residue of group A described above after reaction of said first polymeric material and said third polymeric material, Y represents a residue of said third polymeric material after said reaction of said first and third polymeric materials and X represents a linking atom or group extending between the residues of said first and third polymeric materials. In one preferred embodiment A¹ represents an optionally-substituted phenyl group, x represents a group 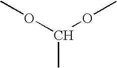
which is bonded via the oxygen atoms to a residue of said third polymeric material. For example, group X may be bonded to the polymer backbone of said third polymeric material.

As described above, said first polymeric material itself may be arranged to change colour according to pH and so for a test material incorporating said first polymeric material said test material need not include any additional indicator means. Preferably, however, said test material comprises a carrier means and an indicator means which is trapped within a matrix defined by the carrier means, but preferably said indicator means is not covalently bonded to the carrier means. In a preferred embodiment, said carrier means includes a hydrogel as described and, preferably, said hydrogel comprise a said second polymeric material as described. In an especially preferred embodiment, said hydrogel comprises cross-linked polyvinylalcohol. Such polyvinylalcohol is preferably cross-linked by said first polymeric material as described.

Preferably, in the method of the first aspect, the pH is assessed on the basis of a change in the visual appearance of said test material. More preferably, the pH is assessed on the basis of the colour of said test material.

The method preferably involves comparing the visual appearance, for example colour, of the test material with a reference means, for example a colour reference means such as a colour chart (or the like) to assess the pH of the substrate or environment.

The test material may be arranged to enable pH information to be obtained directly from it without recourse to any external reference means. For example, said test material may incorporate a said reference means suitably arranged to enable pH information to be obtained directly from the test material.

The method preferably includes the step of recording information relating to the visual appearance of the test material. The colour of the test material may be recorded and/or the pH may be recorded.

Preferably, the method comprises assessing the pH of said substrate or environment; and, subsequently, carrying out another step in dependence upon the pH assessed. For example, when the substrate is a body tissue, for example a wound, the treatment for said tissue is preferably selected in dependence upon the pH assessed.

Said substrate or environment may comprise a solid, liquid or gas. As regards the latter, said test material may be positioned in a gaseous environment to enable the pH of the environment to be assessed. Preferably, said substrate or environment comprises a solid and/or liquid. For example, in a preferred embodiment, it is a body tissue such as a wound which may drain fluid such as exudates or puss.

Said test material may be in sheet form with the area of the main plane of the sheet suitably being less than 1500 cm², preferably less than 1000 cm², more preferably less than 500 cm², especially less than 100 cm². The area may be at least 1 cm². The test material may have a thickness across at least a portion thereof of at least 0.5 mm, preferably at 1 mm, more preferably at least 1.5 mm. The thickness is preferably less than 2 cm, more preferably less than lcm, especially less than 0.6 cm, across substantially its whole extent.

Said first material is preferably arranged to change colour according to pH at first, second, third and fourth positions thereon (which positions are preferably spaced across a surface of the first material) wherein the ratio of the area defined between said four positions (i.e. the area defined by imaginary straight lines joining the four positions to define a quadrilateral shape) to the area of the major surface of said test material is at least 0.5, preferably at least 0.65, more preferably at least 0.8, especially at least 0.9. Preferably, said test material is arranged to change colour across substantially the entire area defined by said four positions, rather than the test material simply being arranged to change colour at points or small regions of the area defined by said four positions. The test material may be arranged to change colour according to pH across substantially its entire major surface.

When said test material is in sheet form and comprises a carrier means and an indicator means, said indicator means is preferably arranged at first, second, third and fourth positions wherein the ratio of the area defined between said four positions to the area of the major surface of said test material is at least 0.5, preferably at least 0.65, more preferably at least 0.8, especially at least 0.9. Especially preferred is the case where indicator means is distributed across substantially the entire area defined by said four positions.

Said test material preferably comprises a solid. It is preferably flexible. It is preferably such that one free end of a sheet thereof can be turned back on itself through at least 90° and preferably 180°. As a consequence, the test material can be contacted with an irregular shaped surface, for example a human or animal body surface, with the material conforming substantially to the surface. Said test material is preferably bio-compatible. It suitably consists of at least 70 wt %, preferably at least 80 wt %, more preferably at least 90 wt %, especially at least 95 wt % water. Advantageously, therefore, said test material may not dehydrate substantially a body tissue to which it may be applied. Said test material may have a pH at a surface used to contact said substrate or environment of less than 7, and, preferably of greater than 3.5. Said pH at said surface may be in the range 4 to 5, preferably 4.5 to 5.

In some cases, a plurality of different test materials may be made available, each being arranged to assess substrates (e.g. wounds) within different pH ranges. An appropriate test material may then be selected in dependence upon the likely pH of a substrate to be assessed.

Said test material may be a component of an assembly. For example, said test material may be affixed or associated with another material, for example so as to define a laminate or the like. Said test material may be a part of a dressing. Said dressing may have a main surface arranged to contact a first area of a tissue (e.g. wound) in use and the test material is such (e.g. by means of an indicator being provided which is suitably dispersed within a carrier) that is arranged to change colour over at least 50%, suitably at least 60%, preferably at least 70%, more preferably at least 80%, especially at least 90%, of the area of said first area so that the pH of individual parts of at least 50% of said first area can be monitored.

When the test material defines a dressing or is a component of a dressing, the test material may facilitate optimum use of dressing material in that the test material may change colour indicating the appropriate time to change the dressing or interact with the wound.

Advantageously, said test material may be arranged to provide a pH map of a substrate which it contacts (e.g. where indicator means is provided and arranged to change colour across a substantial area of the test material). Thus, the test material may display one colour indicative of the pH at a first position which it contacts on the substrate; a second colour indicative of pH at a second position which it contacts on the substrate and so on. Furthermore, as the pH of the substrate (or environment) changes, the colour of the test material changes to indicate the pH change. Thus, the test material allows the pH of a substrate or environment to be tracked over time. The method of the first aspect may include such pH tracking.

Said test material may also be arranged, for example by virtue of it being transparent, to allow colour changes to be observed with the test material in situ. Thus, it may be contacted with a wound and the pH of the wound monitored over time.

Said test material may be arranged to change colour rapidly, for example within 30 seconds, preferably within 15 seconds and, more preferably, within less than 10 seconds. Thus, the test material may, in one embodiment, be contacted with a substrate for the time it takes to change its colour to indicate its pH and may then be removed.

Said test material may include securement means for s securing it relative to said substrate and/or within said environment. Where said test material is used to assess the pH of part of a human or animal body, for example a body tissue such as a wound, said securement means is preferably releasably securable to enable the test material to be releasably secured to said body. Said securement means may comprise tape (or the like) arranged to contact the body for retaining the test material in position.

According to a second aspect of the invention, there is provided a method of making a test material for assessing the pH of a substrate or environment, the method comprising associating an indicator means with a carrier means.

Said test material, said carrier means and said indicator means may have any feature of such means described according to said first aspect.

The method preferably comprises selecting a precursor of said carrier means and causing said precursor to be transformed (e.g. to react) in the presence of said indicator means so that said indicator means becomes associated with, for example incorporated into, said carrier means. In one embodiment, said precursor of said carrier means may be transformed by being cross-linked with a cross-linker means which optionally also acts as said indicator means. In another, preferred embodiment, said precursor is transformed by being cross-linked by a cross-linking means in the presence of an indicator means, additional to said cross-linking means. In this case, the method may be arranged to encapsulate the indicator means within the carrier means without the indicator means being covalently bonded thereto. The method may include the step of derivatising the test material to adjust one or more of its properties, for example to affect a characteristic of the colour change of the test material.

In a further embodiment, the method may comprise causing said precursor of said carrier means to be transformed in the presence of a further active ingredient in order to incorporate said active ingredient into said test material. Said active ingredient may have pharmacological properties; it may be an anti-bacterial agent.

According to a third aspect of the invention, there is provided a method of assessing pH of a substrate or environment, the method comprising contacting the substrate with a test material or introducing the test material into an environment, wherein said test material includes a third polymeric material, cross-linked by a cross-linking means, wherein said cross-linking means incorporates aromatic or heteroaromatic groups.

Said cross-linking means preferably defines a chromophore whereby the test material is arranged to appear coloured under at least some pH conditions. Said cross-linking means preferably incorporates a multiplicity of (preferably at least 4, more preferably at least 8) aromatic and/or heteroaromatic groups. Said cross-linking means may include a phenyl group. Said cross-linking means may include at least one heteroaromatic group, especially a N-containing heteroaromatic group.

According to a fourth aspect of the invention, there is provided a test material as described herein per se.

Said test material of the fourth aspect preferably comprise a hydrogel as described according to said first aspect.

Said test material preferably comprises a carrier means (which is preferably a hydrogel) and an indicator means arranged to change colour according to pH, said indicator means suitably being impregnated in said carrier means. Said indicator means is preferably not covalently bonded to said carrier means.

Said test material of the fourth aspect may have any feature of the test material described in the first, second and third aspects.

According to a fifth aspect of the invention, there is provided a package containing a test material as described herein.

Preferably, said package fully encloses said test material. Said package is preferably sterile and is suitably arranged such that said test material can be applied directly to a wound after removal from the packaging without any need to further sterilise the test material.

According to a sixth aspect of the invention, there is provided the use of a test material as described herein in assessing the pH of a substrate or environment.

In a preferred embodiment, there is provided the use of a test material as described herein for the manufacture of an article for assessing the pH of a substrate comprising a part of a human or animal body.

According to a seventh aspect of the invention, there is provided the use of a said first polymeric material as described herein for assessing the pH of a substrate or environment.

Any feature of any aspect of any invention or embodiment described herein may be combined with any feature of any aspect of any other invention or embodiment described herein mutatis mutandis.

Specific embodiments of the invention will now be described, by way of example.

In general terms, the pH of a wound may be assessed using a hydrogel film which changes colour in dependence upon pH. Such wound pH information may be used to facilitate selection of the appropriate treatment to which the wound should be subjected. The hydrogel can be sterilised in an autoclave and loaded with antibacterial/antiseptic agents to provide a wound dressing which will indicate the pH of wound exudates in a non-invasive and simple manner.

Further details are provided in the examples which follow. The examples illustrate how a hydrogel film may be prepared (Examples 1 and 8) which changes colour (Example 2); how the colour change of the film may be enhanced and adjusted (Examples 3 to 5); how conventional acid/base indicators may be incorporated into a hydrogel film (Example 6); and how the film may be rendered anti-bacterial (Example 7).

### EXAMPLE 1

### General Method of Preparing Hydrogel Film

Step (a)—Preparation of poly (1,4-di(4-(N-methylpyridinyl))-2,3-di(4-(1-formylphenyl)butylidene

This was prepared as described in Example 1 of PCT/GB97/02529, the contents of which are incorporated herein by reference. In the method, an aqueous solution of greater than 1 wt % of 4-(4-formylphenylethenyl)-1-methylpyridinium methosulphonate (SbQ) is prepared by mixing the SbQ with water at ambient temperature. Under such conditions, the SbQ molecules form aggregates. The solution was then exposed to ultraviolet light. This results in a photochemical reaction between the carbon-carbon double bonds of adjacent 4-(4-formylphenylethenyl)-1-methylpyridinium methosulphate molecules (VIII) in the aggregate, producing a polymer, poly (1,4-di(4-(N-methylpyridinyl))-2, 3-di(4-(1-formylphenyl)butylidene methosulphonate (IX), as shown in the reaction scheme below. It should be appreciated that the anions of compounds VIII and IX have been omitted in the interests of clarity.

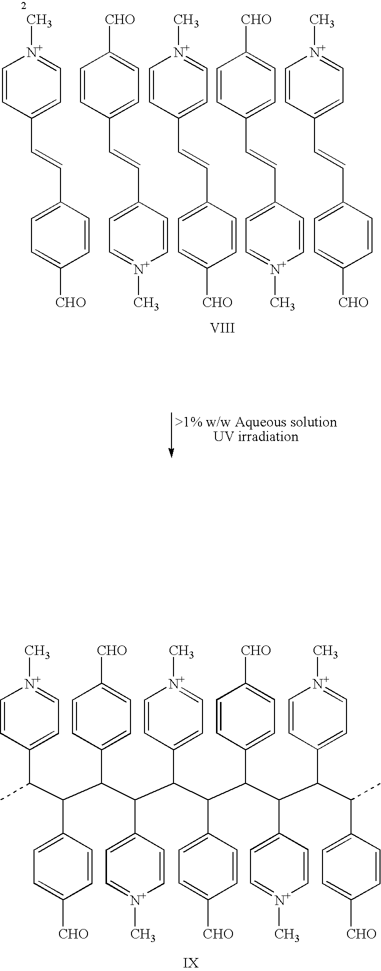

Step (b)

A predetermined amount of 88% hydrolysed poly(vinylalcohol) of molecular weight 300,000 was dissolved in water by heating to 60° C. for 6 hours. Then this is allowed to cool.

Step (c)

A solution comprising 8 wt % of poly(vinylalcohol) of step (b) and 0.33 wt % of the butylidene polymer of step (a) was prepared in distilled water and an acid catalyst (HCl) added to lower the pH of the solution to less than 2.5. The solution was then poured into a glass petri dish (or onto a stainless steel substrate) to a depth of 2 mm thickness. This was allowed to air dry for 24 hours. Thereafter, the film was peeled from the substrate and vacuum dried at 50° C. for 1 hour.

After addition of the acid catalyst as aforesaid, the mixture polymerises, whereby the butylidene polymer of step (a) cross-links the poly(vinylalcohol) according to the scheme below.

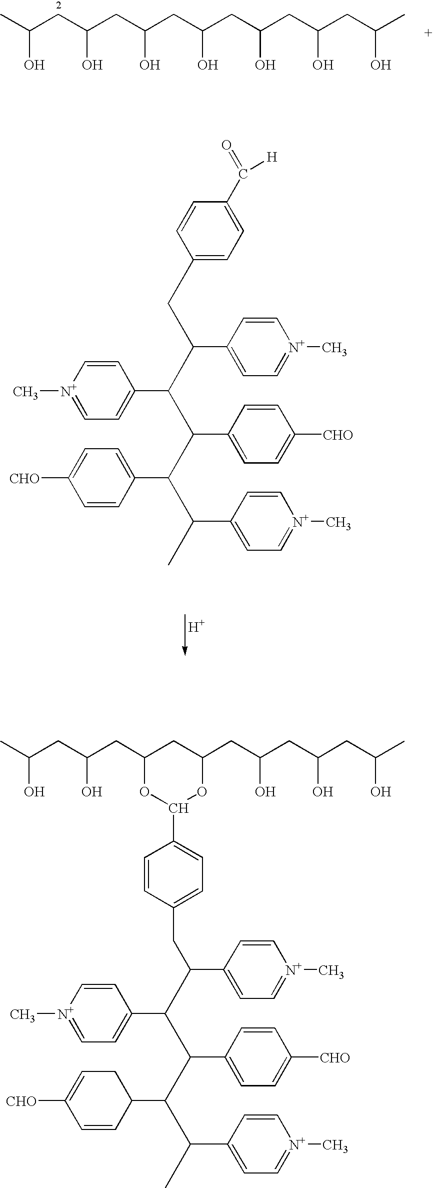

### EXAMPLE 2

### Change of Colour of Film with pH

The film of Example 1 was re-hydrated in de-ionised or distilled water and placed in contact with moist surfaces of known pH. On contact with a new surface the film changes colour in 2 to 4 seconds to indicate the pH of the surface by the colour adopted. The film is pale yellow at pH 1 to 2; changes to shades of orange up to pH 7; then goes through green and blues as the pH is raised through the alkaline region.

### EXAMPLE 3

### Enhancing Colour Change of Film

Dried film prepared as described in Example 1 was immersed in 4M NaOH for 16 hours. (Other alkalis can be used if desired). This is believed to cause conversion of aldehyde groups on the residue of the butylidene polymer to carboxylate groups and the film turns dark blue. On immersion in 7% hydrochloric acid, the colour of the film changes to a very pale yellow. In general terms, the aforementioned acid is used to neutralise the alkali. Then, the film is washed with distilled water to remove acid.

The film prepared may be assessed as described in Example 2 in which it is found that the colour change with pH is intensified.

### EXAMPLE 4

### Derivatisation of Butylidene Polymer

The dry film of Example 1 was immersed in a solution of the butylidene polymer of step (a) in methanol. (Other solvents such as acetone or any other solvent which will dissolve the butylidene polymer but not dissolve, swell or penetrate the dry film may be used). This ensures that the reaction of the dry film with the butylidene polymer occurs only at the surface and not in the bulk of the film. The mixture was then acidified to a pH of less than 2.5 using concentrated hydrochloric acid and the reaction allowed to continue for 1 hour. The film was then removed from the solution and washed with methanol. The film was then treated as described in Example 3 to convert the aldehyde groups on the butylidene polymer (both in the bulk and at the surface) to carboxylic acid groups. When the film prepared is treated as in Example 2, a more intense colour change, compared to that with the Example 1 embodiment, is observed.

### EXAMPLE 5

### Chemical Modification of Hydrogel Film

The films prepared and treated as described in Examples 1 and 2 may be subjected to a range of reactions to modify them, with the result often being a different colour change. For example, reacting hydroxyl groups on a poly(vinyl alcohol) with urea, in an acidic solution, produces a more intense green colour in the alkaline pH region.

### EXAMPLE 6

### Preparation of Film Incorporating Universal Indicator

33 ml of a solution comprising 10 wt % of poly(vinylalcohol) of Example 1, step (b) and 0.5 wt % of the butylidene polymer of Example 1, step (a) was selected together with 1 ml of Universal indicator solution (an approximate 1 wt % solution in iso-propanol) Gelation was initiated by addition of 0.5 ml of 20% HCl solution and the mixture poured into a Petri dish to form a film which was allowed to cure and air dry. The resultant film is sensitive to pH, as indicated by a colour change of the gel, with the pH range 1-14.

The film may be used as a dressing because of its high water content. It may be placed on an open wound to monitor the pH of the wound by means of a colour change.

### EXAMPLE 7

### Incorporation of Anti-Bacterial

The procedure of Example 1 was followed except that, before the addition of the acid catalyst in step (c), 0.5 wt % of an antibacterial agent (neomycin sulphate or cetrimide) was added. The acid catalyst was then added and the preparation of the film was continued as described in step (c). The film still changes colour with pH as described in Example 2 and may be further treated as described in Examples 3 to 5.

Advantageously, the film prepared may be used to define an anti-bacterial dressing or part of such a dressing which automatically is able to provide pH information on the state of the wound to which it is applied.

An antibacterial agent may also be incorporated into the film of Example 6.

### EXMAPLE 8

### Use of Alternative Poly(vinylalcohols)

The process of Example 1 was repeated with poly(vinylalcohols) of different degrees of hydrolysis and/or different molecular weights. It was found that the strength of films prepared is affected by the aforementioned variables.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A method of assessing the pH of a substrate or environment, the method comprising contacting the substrate with a test material or introducing the test material into an environment, wherein said test material is arranged to change colour according to pH.

2. A method according to claim 1, wherein said substrate or environment is a tissue of a human or animal body, and said test material is at least a part of a dressing having a main surface arranged to contact a said tissue wherein the test material is arranged to contact a first area of a said tissue and the test material is such that it is arranged to change colour over at least 50% of the area of said first area so that the pH of individual elements of at least 50% of said first area can be monitored.

3. A method according to claim 2, wherein said test material is a hydrogel.

4. A method according to claim 2, wherein said material comprises a carrier means and an indicator means arranged to change colour according to pH.

5. A method according to claim 4, wherein said carrier means and said indicator means are covalently bonded to one another.

6. A method according to claim 4, wherein said indicator means is impregnated in said carrier means and trapped therein in a matrix defined by said carrier means.

7. A method according to claim 2, wherein said test material includes at least 0.01 wt % and less than 3 wt % of said indicator means.

8. A method according to claim 4, wherein said carrier means comprises a natural or synthetic polymer or a residue thereof in the event that said indicator means is covalently bonded to the carrier means; and said indicator means comprises a natural or synthetic material or a residue thereof in the event said indicator means is covalently bonded to said carrier means.

9. A method according to claim 1, wherein said test material is in sheet form and is arranged to change colour according to pH at first, second, third and fourth positions thereon, wherein the ratio of the area defined between said four positions to the area of the main surface of the sheet is at least 0.5.

10. A method according to claim 2, wherein said test material includes a second polymeric material comprising a third polymeric material which is cross-linked by a cross-linking means.

11. A method according to claim 10, wherein said third polymeric material is a polyvinyl polymer or a copolymer comprising a polyvinyl repeat unit.

12. A method according to claim 10, wherein said the third polymeric material is selected from optionally substituted polyvinyl alcohol, polyvinyl acetate, polyalkalene glycols and collagen.

13. A method according to claim 10, wherein said second polymeric material includes cross-linked polyvinyl alcohol or a copolymer thereof.

14. A method according to claim 10, wherein said second polymeric material includes a moiety of formula I: 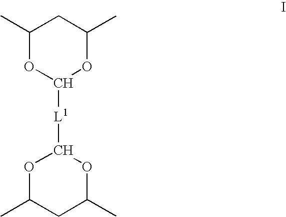
wherein L¹ is a residue of said cross-linking means.

15. A method according to claim 10, wherein said cross-linking means comprises:
(a) a first polymeric material having a repeat unit of formula 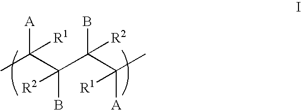
wherein A and B are the same or different, are selected from optionally-substituted aromatic and heteroaromatic groups and at least one comprises a relatively polar atom or group and R¹ and R² independently comprise relatively non-polar atoms or groups; or
(b) a first polymeric material prepared or preparable by providing a compound of general formula 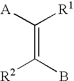
wherein A, B, R¹ and R² are as described above, in an aqueous solvent and causing the groups C=C in said compound to react with one another to form said first polymeric material.

16. A method according to claim 1, wherein said test material comprises a carrier means and an indicator means which is trapped within a matrix defined by the carrier means wherein said indicator means is not covalently bonded to the carrier means.

17. A method according to claim 2, which includes the step of comparing the visual appearance of the test material with a reference means; or the test material may be arranged to enable pH information to be obtained directly from it without recourse to any external reference means.

18. A method according to claim 2, wherein the method comprises assessing the pH of said substrate or environment; and, subsequently, carrying out another step in dependence upon the pH assessed.

19. A method according to claim 18, wherein said substrate is a tissue of the human or animal body and a subsequent treatment of said body is selected in dependence upon the pH assessed.

20. A method according to claim 1, wherein said test material is part of a dressing for the human or animal body.

21. A method according to claim 1, wherein said test material is arranged to provide a pH map of a substrate which it contacts.

22. A method according to any claim 2, wherein said test material is arranged, by virtue of it being transparent, to allow colour changes to be observed with the test material in situ.

23. A method according to claim 2, wherein said test material includes securement means for securing it relative to a said substrate wherein said test material is used to assess the pH of part of a human or animal body.

24. (canceled)

25. (canceled)

26. (canceled)

27. (canceled)

28. (canceled)

29. A method of assessing pH of a substrate or environment, the method comprising contacting the substrate with a test material or introducing the test material into an environment, wherein said test material includes a third polymeric material, cross-linked by a cross-linking means, wherein said cross-linking means incorporates aromatic or hetero-aromatic groups.

30. (canceled)

31. A package which contains a test material in a sterile environment wherein said test material, comprises a carrier means in a form of a hydrogel and an indicator means arranged to change colour according to pH.

32. (canceled)

33. (canceled)

34. (canceled)

35. (canceled)

36. (canceled)

37. The use of a test material according to claim 31 in assessing the pH of a substrate or environment.

38. A method of assessing the pH of a tissue of a human or animal body, the method comprising contacting said tissue with a test material which comprises a hydrogel comprising a carrier means and an indicator means arranged to change colour according to pH, wherein said test material is in sheet form and is arranged to change colour independently according to pH at first, second, third and fourth positions thereon, wherein the ratio of the area defined between said first, second, third and fourth positions to the area of a major surface of said test material is at least 0.5, wherein said test material is arranged to display a colour indicative of the pH at a first position which it contacts and to display a colour indicative of the pH at another position it contacts so that a pH map of a tissue contacted by said test material can be defined.

39. A test material which comprises a hydrogel comprising a carrier means and an indicator means arranged to change colour according to pH, wherein said test material is in sheet form and is arranged to change colour independently according to pH at first, second, third and fourth positions thereon, wherein the ratio of the area defined between said first, second, third and fourth positions to the area of a major surface of said test material is at least 0.5, wherein said test material is arranged to display a colour indicative of the pH at a first position which it contacts in use and to display a colour indicative of the pH at another position it contacts in use so that a pH map of a tissue contacted in use by said test material can be defined.
